(19) United States
(12) Patent Application Publication
(10) Pub. No.: US 2022/0257454 A1
(43) Pub. Date: Aug. 18, 2022

(54) MASSAGE APPARATUS INCLUDING MASSAGE UNIT CAP ABLE OF PROVIDING FEELING OF WARMTH

(71) Applicant: BODYFRIEND CO., LTD., SEOUL (KR)

(72) Inventors: Deok Hyun KONG, Seoul (KR); Yong Ju KWON, Seoul (KR); Gi Won UM, Bucheonsi (KR); Ryang Min LEE, Incheon (KR)

(21) Appl. No.: 17/625,798

(22) PCT Filed: Jun. 29, 2020
(86) PCT No.: PCT/KR2020/008440

§ 371 Date: Jan. 10, 2022

(30) Foreign Application Priority Data
Jul. 10, 2019 (KR) 10-2019-0083333
Jul. 10, 2019 (KR) 10-2019-0140098
Mar. 18, 2020 (KR) 10-2020-0033114

(51) Int. Cl. A61H 15/02; A61H 15/00

(52) U.S. Cl. CPC A61H 15/02; A61H 15/0078; A61H 2201/0207; A61H 2201/0235; A61H 2015/0042; A61H 2201/5082; A61H 2201/1654

(57) ABSTRACT

The present disclosure provides a massage apparatus including a massage unit capable of providing a feeling of warmth and includes a body massage module which provides a massage function to at least a part of a user's body. The body massage module includes an actuator, which moves the body massage module, and a massage unit which mechanically stimulates the user's body. The massage unit includes a massage ball which provides a feeling of warmth to a user, and the massage ball includes a heating member, which adjusts a temperature of the massage ball, a support shaft member, which fixes the heating member, a cushion member, which surrounds the support shaft member and provides a feeling of a soft massage to the user, and an insert member which maintains a shape of the cushion member.

### TECHNICAL FIELD

The present disclosure relates to a massage apparatus.

### BACKGROUND ART

Massage is medical assistance which adjusts the irregularity of a subject's body, helps blood circulation, and relieves fatigue of a subject by applying various types of mechanical stimuli, such as kneading, pressing, pulling, tapping, or moving, to some portions of the subject's body.

An increase in demand for massage causes an increase in demand for massage apparatuses or massage devices which provide an artificial massage function due to economic and time reasons. That is, according to the increase in demand to relieve fatigue or stress through the massage while loosening tight muscles, massage apparatuses operating in a timely and cost effective manner are on the market. Any type of unit, device, or apparatus which performs a massage without a separate massage therapist using a mechanical apparatus is referred to as a massage apparatus.

The massage apparatus includes a massage ball and may stimulate a user's body using the massage ball, and research to provide a feeling of warmth for an effective massage is being conducted.

### Technical Problem

The present disclosure is directed to providing a massage apparatus including a massage unit capable of providing a feeling of warmth.

### Technical Solution

One aspect of the present disclosure provides a massage apparatus including a body massage module which provides a massage function to at least a part of a user's body, wherein the body massage module includes an actuator, which moves the body massage module, and a massage unit, which mechanically stimulates the user's body, the massage unit includes a massage ball which provides a feeling of warmth to a user, the massage ball includes a heating member, which adjusts a temperature of the massage ball, a support shaft member, which fixes the heating member, a cushion member, which surrounds the support shaft member and provides a feeling of a soft massage to the user, an insert member, which maintains a shape of the cushion member, an inner warming member, which receives heat from the heating member, and a temperature detection sensor unit which senses a temperature of the inner warming member, and the control unit controls a temperature of the heating member on the basis of temperature information obtained from the temperature detection sensor unit. In addition, when the temperature information obtained from the temperature detection sensor unit is greater than a predetermined threshold value, the control unit may block power being supplied to the heating member.

The massage apparatus may further include a physical current blocking unit which is provided in the inner warming member and blocks a current being supplied to the heating member according to the temperature of the inner warming member.

The physical current blocking unit may be formed as a bi-metal module.

### Advantageous Effects

In a massage apparatus according to one embodiment of the present disclosure, a massage ball includes a heating member, and thus a massage and a feeling of warmth are provided to a user at the same time.

Effectives which can be obtained through the present disclosure are not limited the above-described effects, and other effects, which are not described above, will be clearly understood through the following description by those skilled in the art included in the present disclosure.

### DESCRIPTION OF DRAWINGS

FIG. 1 is a view for describing a massage apparatus according to one embodiment of the present disclosure.

FIG. 2 is a view for describing components of the massage apparatus according to one embodiment of the present disclosure.

FIG. 3 is a view for describing a main frame according to one embodiment of the present disclosure.

FIG. 4 is a view illustrating a body massage module according to one embodiment of the present disclosure.

FIG. 5 is a perspective view illustrating an assembled massage unit according to a first embodiment of the present disclosure.

FIG. 6 is an exploded perspective view illustrating a disassembled message unit according to the first embodiment of the present disclosure.

FIG. 7 is a cross-sectional view illustrating a massage ball according to the first embodiment of the present disclosure when viewed from above.

FIG. 8 is a set of cross-sectional views illustrating the massage ball according to the first embodiment of the present disclosure when viewed from the side.

FIG. 9 is a set of perspective views illustrating an assembled massage unit according to a second embodiment of the present disclosure.

FIG. 10 is an exploded perspective view illustrating a disassembled massage unit according to the second embodiment of the present disclosure.

FIG. 11 is set of perspective views illustrating an assembled massage unit according to a third embodiment of the present disclosure.

FIG. 12 is an exploded perspective view illustrating a disassembled massage unit according to the third embodiment of the present disclosure.

FIG. 13 is a set of views illustrating an insert member according to another embodiment of the present disclosure.

FIG. 14 is a view for describing a method of controlling a temperature of a massage ball according to another embodiment of the present disclosure.

FIG. 15 is a view for describing an inner warming member (820) in which a temperature detection sensor unit (822) and a physical current blocking unit (824) are provided according to one embodiment of the present disclosure.

### BEST MODES OF THE DISCLOSURE

A massage apparatus includes a body massage module which provides a massage function to at least a part of a user's body, wherein the body massage module includes an actuator, which moves the body massage module, and a massage unit, which mechanically stimulates the user's body, the massage unit includes a massage ball which provides a feeling of warmth to a user, the massage ball includes a heating member, which adjusts a temperature of the massage ball, a support shaft member, which fixes the heating member, a cushion member, which surrounds the support shaft member and provides a feeling of a soft massage to the user, an insert member, which maintains a shape of the cushion member, an inner warming member, which receives heat from the heating member, and a temperature detection sensor unit which senses a temperature of the inner warming member, and the control unit controls a temperature of the heating member on the basis of temperature information obtained from the temperature detection sensor unit.

### Modes of the Disclosure

Objectives, features, and advantages of the present disclosure will become more apparent by the following embodiments described in conjunction with the accompanying drawings. The following structural and functional descriptions are only exemplified for describing the embodiments according to the concept of the present disclosure, and the embodiments according to the concept of the present disclosure may be implemented in various forms and should not be interpreted as being limited to the embodiments described in the present specification or application.

While the disclosure is susceptible to various modifications and alternative forms, specific embodiments thereof will be shown by way of example in the drawings and will herein be described in detail. It should be understood, however, that there is no intent to limit the disclosure to the particular forms disclosed, and the disclosure is to cover all modifications, equivalents, and alternatives falling within the spirit and scope of the disclosure.

Although the terms first, second, and/or the like may be used herein to describe various elements of the present disclosure, such elements are not to be construed as limited by the terms. The terms are only used to distinguish one element from another. For example, a first element could be called a second element, and a second element could be called a first element without departing from the scope of the present disclosure.

It should be understood that, when an element is referred to as being “connected” or “coupled” to another element, the element may be directly connected or coupled to another element, or intervening elements may be present. In contrast, when an element is referred to as being “directly connected” or “directly coupled” to another element, there are no intervening elements. Other words, that is, “between” and “directly between,” or “adjacent” and “directly adjacent,” and the like, which are used to describe relationships between elements should be interpreted in a like fashion.

The terms used in the present specification are only used for the purpose of describing specific embodiments and are not intended to limit the present disclosure. The singular forms include the plural forms, unless the context clearly indicates otherwise. It should be understood that the terms “comprises,” “comprising,” “includes,” and/or “including” used in the specification specify the presence of stated features, integers, steps, operations, elements, parts, or groups thereof, and do not preclude the presence or addition of one or more other features, integers, steps, operations, elements, parts, and/or groups thereof.

Unless otherwise defined, all terms including technical and scientific terms used herein should be interpreted as is customary in the art to which the present disclosure belongs. It should be further understood that terms in common usage should also be interpreted as is customary in the relevant art and not in an idealized or overly formal sense unless expressly so defined herein.

In the present specification, an actuator denotes a component capable of providing a driving force. For example, the actuator may include a motor, a linear motor, an electronic motor, a direct current (DC) motor, an alternating current (AC) motor, a linear actuator, an electric actuator, or the like, but is not limited thereto.

According to one embodiment of the present disclosure, a massage apparatus may be referred to as a massage apparatus including a massage unit having a body massage unit and a leg massage unit.

In addition, according to another embodiment, a body massage unit and a leg massage unit may be present as separate apparatuses (for example, a body massage apparatus and a leg massage apparatus), and the massage apparatus may be referred to as the body massage apparatus or leg massage apparatus.

Hereinafter, embodiments of the present disclosure will be described in more detail with reference to the accompanying drawings.

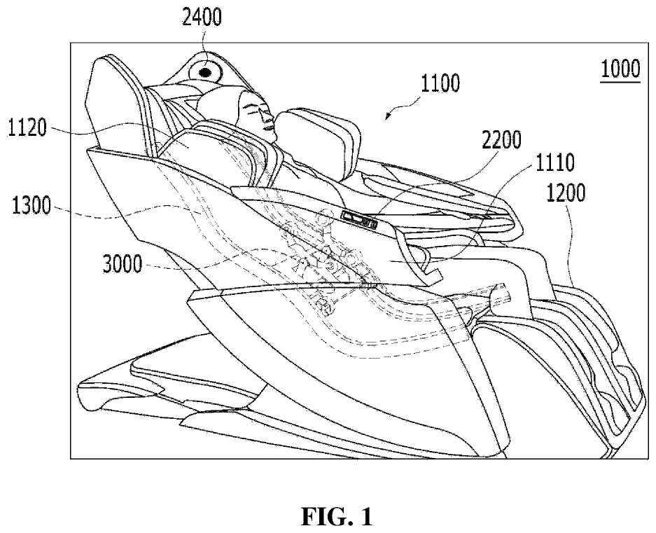

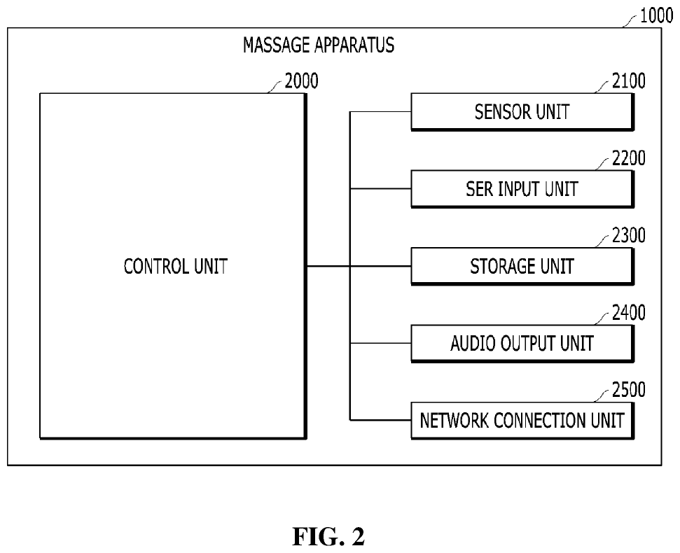

FIG. 1 is a view for describing a massage apparatus according to one embodiment of the present disclosure. In addition, FIG. 2 is a view for describing components of the massage apparatus according to one embodiment of the present disclosure.

Referring to FIGS. 1 and 2, a massage apparatus 1000 according to one embodiment of the present disclosure may include at least one of a control unit 2000, a sensor unit 2100, a user input unit 2200, a storage unit 2300, an audio output unit 2400, a network connection unit 2500, and massage units 1100 and 1200.

The control unit 2000 may control the operation of the massage apparatus 1000. The control unit 2000 may be implemented as one processor or plurality of processors, but is not limited thereto, and may be implemented in various ways.

When the control unit 2000 is implemented as the plurality of processors, at least some of the plurality of processors may be physically spaced a distance from the remaining processors. In addition, the control unit 2000 is not limited thereto and may be implemented as one of various forms.

According to one embodiment of the present disclosure, the control unit 2000 may include the operation of the massage apparatus 1000. For example, the massage apparatus 1000 may include a plurality of actuators, and the massage apparatus 1000 may control the operations of the plurality of actuators to control the operation of the massage apparatus 1000. For example, the massage apparatus 1000 may include at least one of a massage module moving actuator, at least one actuator included in a massage module, a back angle actuator, a leg angle actuator, a foot massage actuator, a leg length adjusting actuator, and a sliding actuator, and the control unit 2000 may control the operation of the massage apparatus 1000 by controlling the at least one thereof.

The sensor unit 2100 may obtain various types of information using at least one sensor. For example, the sensor may include a pressure sensor, an infrared sensor, a light emitting diode (LED) sensor, and the like but is not limited thereto.

In addition, the sensor unit 2100 may include a biometric information acquisition sensor. The biometric information acquisition sensor may obtain fingerprint information, face information, voice information, iris information, weight information, electrocardiogram information, and the like but is not limited thereto, and may obtain various types of biometric information.

According to another embodiment of the present disclosure, the massage apparatus 1000 may detect a contact area and/or contact position with a user using sensors. For example, the massage apparatus 1000 may obtain a user's shoulder position information using the sensor unit 2100. The massage apparatus 1000 may provide a customized massage on the basis of the obtained information.

The user input unit 2200 may receive any type of input from the user. The massage apparatus 1000 may include the user input unit 2200 for receiving a command related to operation control from the user, and the user input unit 2200 may be implemented in one of various forms. For example, the user input unit 2200 may be provided in the massage apparatus 1000 as a user input module type (for example, remote controller). In addition, the user input unit 2200 may be integrally provided with the body massage unit 1100 or the leg massage unit 1200 but is not limited thereto.

The massage apparatus 1000 may receive various commands from the user through the user input unit 2200. For example, the massage apparatus 1000 may receive any type of command related to selection of a massage module, selection of a massage type, selection of massage intensity, selection of massage time, selection of a massage portion, selection of a position and operation of the body massage unit 1100, selection of power On-Off of the massage apparatus 1000, selection of whether to operate a warming function, selection related to sound source playback, and the like but is not limited thereto.

According to another embodiment of the present disclosure, the user input unit 2200 my include hot key type buttons and/or selection buttons for selecting a direction, cancelling, and executing an input according to a preset user-setting function, self-preset function, or the like.

The user input unit 2200 may be implemented as a key pad, a dome switch, a touchpad (pressure/capacitance), a jog wheel, a jog switch, or the like but is not limited thereto. In addition, the user input unit 2200 may receive a command through the utterance of the user on the basis of voice recognition technology.

According to one embodiment of the present disclosure, the user input unit 2200 may include a display for displaying an operation situation of the massage apparatus 1000 or current state of the user. In this case, the display may include at least one of a liquid crystal display (LCD), a thin film transistor-liquid crystal display (TFT LCD), an organic light-emitting diode (OLED), a flexible display, a three-dimensional (3D) display but is not limited thereto.

The storage unit 2300 may store various types of information related to the massage apparatus 1000. For example, the storage unit 2300 may store massage control information and personal authentication information but is not limited thereto.

The storage unit 2300 may be implemented as a non-volatile storage medium capable of continuously storing any type of data. For example, the storage unit 2300 may include a disk, an optical disc, a magneto-optical storage device, a flash memory, and/or a storage device on the basis of a battery-backup memory but is not limited thereto.

In addition, the storage unit 2300 may include a memory. The memory may be a volatile storage device, such as a random access memory (RAM) including a dynamic random access memory (DRAM) and a static random access memory (SRAM), which are directly accessed by a processor and in which stored information is instantaneously erased when a power source is turned off but is not limited thereto. The memory may be operated by the control unit 2000.

The audio output unit 2400 may provide any type of audio output to the user. For example, the audio output unit 2400 may output a sound source and/or binaural beat optimized to a massage pattern provided by the massage apparatus 1000 to the user to stimulate a brain of the user. The audio output unit 2400 may output a sound signal received through a network (not shown) or stored in an internal/external storage medium (not shown). For example, the audio output unit 2400 may be connected to a user's terminal through a network (for example, Bluetooth connection) and may output a sound source according to control of the user's terminal. In addition, the audio output unit 2400 may output any type of sound signal generated due to operation of the massage apparatus 1000.

According to one embodiment of the present disclosure, the audio output unit 2400 may be provided at various positions. For example, the audio output unit 2400 may include a plurality of output units such as an upper end audio output unit disposed at an upper end of a seat part in contact with the user, front audio output units attached to front ends of arm massage units disposed at the left and right of the seat part, and/or rear audio output units attached to rear ends of the arm massage units but is not limited thereto. In this case, the massage apparatus 1000 may provide 3D sound such as 5.1 channel but is not limited thereto.

The network connection unit 2500 may communicate with an external massage apparatus and/or user's terminal through any type of network. The network connection unit 2500 may include a wired/wireless access module for network access.

As a wireless access technology, for example, wireless local area network (wireless LAN, Wi-Fi), wireless broadband (WiBro), world interoperability for microwave access (WiMAX), high speed downlink packet access (HSDPA), or the like may be used. As a wired access technology, for example, digital subscriber line (XDSL), fibers to the home (FTTH), power line communication (PLC), or the like may be used.

In addition, the network connection unit 2500 may include a short range communication module to transmit/receive data to/from any type of apparatus/terminal positioned within a short distance. For example, as a short range communication technology, Bluetooth, radio frequency identification (RFID), infrared data association (IrDA), ultra-wideband (UWB), ZigBee, or the like may be used.

In each of the massage units 1100 and 1200, a portion for accommodating at least a part of the user's body may be formed, and the massage units 1100 and 1200 may massage the user's body. The massage units 1100 and 1200 may include a body massage unit 1100, which massages the user's body, and a leg massage unit 1200 which massages a user's leg.

The body massage unit 1100 may massage at least a part of the user's body. Any type of space for accommodating the user may be formed in the body massage unit 1100. The body massage unit 1100 may have a space having a shape corresponding to a shape of the user's body. For example, as illustrated in FIG. 1, the body massage unit 1100 may be formed in a chair shape capable of accommodating the user's whole body or a part of the user's body.

A portion, which is in contact with a floor surface, of the body massage unit 1100 may be formed of any material to increase a frictional force or may include any member (for example, a non-slip pad) to increase a frictional force and may include wheels to improve the mobility of the massage apparatus 1000.

The body massage unit 1100 may include a head contact portion which is in contact with a user's head area, a back contact portion which is in contact with a user's back area, a hip contact portion which is in contact with a user's hip area, an arm massage unit 1110 which accommodates a user's arm areas, and a shoulder massage unit 1120 which massages a user's shoulder areas but is not limited thereto, and may include user contact portions having various shapes.

At least a part of the body massage unit 1100 may move in a sliding manner. For example, when the body massage unit 1100 starts a massage, at least a part of the body massage unit 1100 may move forward in the sliding manner. In this case, the body massage unit 1100 may be inclined in a rearward direction. As a result, the body massage unit 1100 may provide a massage in a state of being inclined in the rearward direction.

According to one embodiment of the present disclosure, the massage apparatus 1000 may include one or more air cells (not shown). The air cells may be positioned in a portion for a user's pelvis, the arm massage unit 1110, the shoulder massage unit 1120, the leg massage unit 1200, and the like but is not limited thereto, and may be disposed in various portions of the massage apparatus 1000.

The massage apparatus 1000 may include an air supply unit, the air supply unit may supply air to the air cell to inflate the air cell according to control of the control unit. The air supply unit may be positioned inside or outside the body massage unit 1100 and may be positioned in the leg massage unit 1200.

The body massage unit 1100 may include at least one of a main frame 1300 constituting a frame of the body massage unit 1100 and a body massage module 3000 which provides a massage function to at least a part of the user's body.

The body massage module 3000 may be provided in the body massage unit 1100 to provide any type of mechanical stimulation to the user accommodated in the body massage unit 1100. As illustrated in FIG. 1, the body massage module 3000 may move along the main frame 1300 provided in the body massage unit 1100.

For example, the main frame 1300 of the body massage unit 1100 may include a rack gear, and the body massage module 3000 may mechanically stimulate various parts of the user's body while moving along the rack gear.

The main frame 1300 constitutes a frame of an internal structure of the body massage unit 1100 and may be formed of a metal material, a plastic material, or the like. For example, the main frame 1300 may be formed of iron, an alloy, steel, or the like but is not limited thereto, and may be formed of one of various hard materials.

The above-described components included in the massage apparatus 1000 are only exemplary, and the massage apparatus 1000 may be include various components in addition to the above-described components.

In addition, a shape and a structure of the massage apparatus 1000 illustrated in FIG. 1 are only exemplary, and various types of massage apparatuses 1000 may also be included in the range of the present disclosure as long as the massage apparatuses do not depart from the scope of rights defined by the appended claims of the present disclosure.

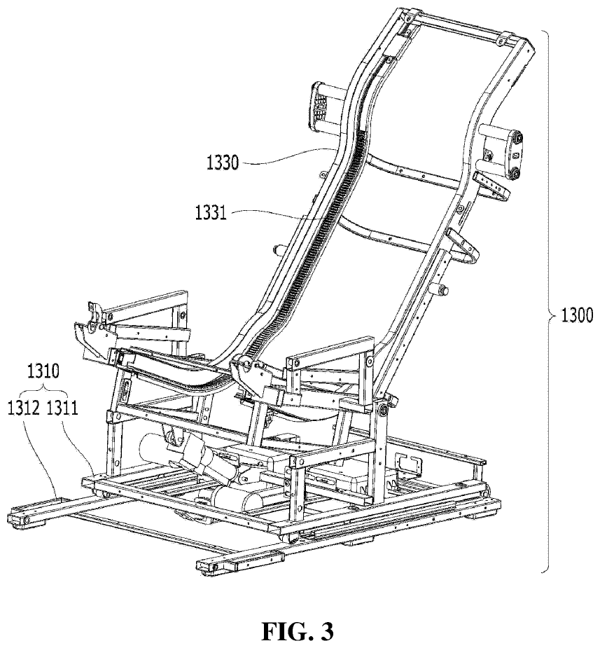

FIG. 3 is a view for describing the main frame according to one embodiment of the present disclosure.

According to one embodiment of the present disclosure, the main frame 1300 may include an upper frame 1330, which includes the body massage module 3000, and a base frame 1310 which supports the upper frame 1330. The upper frame 1330 and the base frame 1310 may be separated, may be formed of respective frames, but may also be formed of one frame.

Rack gears 1331 may be provided on at least a part of the upper frame 1330. The rack gear 1331 is a member for guiding vertical movement of the body massage module 3000 and may include a plurality of valley portions and a plurality of ridge portions.

According to one embodiment of the present disclosure, the rack gears 1331 may be formed at both side portions of the upper frame 1330 to face each other, and the body massage module 3000 may move along the rack gears 1331.

For example, the body massage module 3000 may include gears meshing with the rack gears 1331, and as the gears are rotated by an actuator provided in the body massage module 3000, the body massage module 3000 may be moved upward or downward.

The rack gear 1331 may be formed of a metal material or plastic material. For example, the rack gear 1331 may be formed of iron, steel, an alloy, reinforced plastic, a melamine resin, a phenolic resin, or the like but is not limited thereto.

The upper frame 1330 may be implemented in one of various shapes. For example, the upper frame 1330 may be divided into an I frame, S frame, L frame, S&L frame, or double S&L frame according to a shape but is not limited thereto.

The I frame means a frame in which at least a part of the upper frame 1330 is formed in an “I” shape. When the body massage module 3000 is provided on the I frame, the body massage module 3000 may linearly move along the I frame.

The S frame means a frame in which at least a part of the upper frame 1330 has a curved shape such as “S.” The L frame denotes a frame in which at least a part of the upper frame 1330 has a bent shape such as “L,” the S&L frame means a frame having both of a curved shape such as “S” and a bent shape such as “L,” and the double S&L frame means a frame in which a bent shape such as “L” is formed and two portions are formed in curved shapes such as “S.”

The base frame 1310 means a portion which supports the upper frame 1330 of the main frame 1300 and is in contact with the floor surface. The base frame 1310 may include an upper base frame 1311 and a lower base frame 1312.

The upper base frame 1311 may support the upper frame 1330, and the lower base frame 1312 may be in contact with the floor surface. In addition, the upper base frame 1311 may be positioned to be in contact with the lower base frame 1312.

According to one embodiment of the present disclosure, the upper base frame 1311 may move along the lower base frame 1312. For example, the upper base frame 1311 may move forward or rearward along the lower base frame 1312 in a sliding manner. In this case, the upper frame 1330 may be connected to the upper base frame 1311 and moved according to movement of the upper base frame 1311.

For example, when the upper base frame 1311 moves forward, the upper frame 1330 may also move forward, and when the upper base frame 1311 moves rearward, the upper frame 1330 may also move rearward. Accordingly, the body massage unit 1100 may be allowed to move in a sliding manner.

Specifically, in order to allow movement of the upper base frame 1311, a moving wheel may be provided under the upper base frame 1311. In addition, a guide member capable of guiding the moving wheel may be provided on the lower base frame 1312. Since the moving wheel provided under the upper base frame 1311 moves along the guide member provided on the lower base frame 1312, forward movement or rearward movement thereof may be allowed.

According to another embodiment of the present disclosure, an upper base frame 1311 and a lower base frame 1312 may be formed as one frame instead of being separated frames, and the upper base frame 1311 may be fixed to the lower base frame 1312 instead of moving along the lower base frame 1312. For example, the upper base frame 1311 may be fixed instead of sliding forward or rearward.

Specifically, a moving wheel may not be provided under the upper base frame 1311, and a fixing member for fixing the upper base frame 1311 and the lower base frame 1312 may be provided between the upper base frame 1311 and the lower base frame 1312. In addition, the upper base frame 1311 and the lower base frame 1312 may be integrally formed.

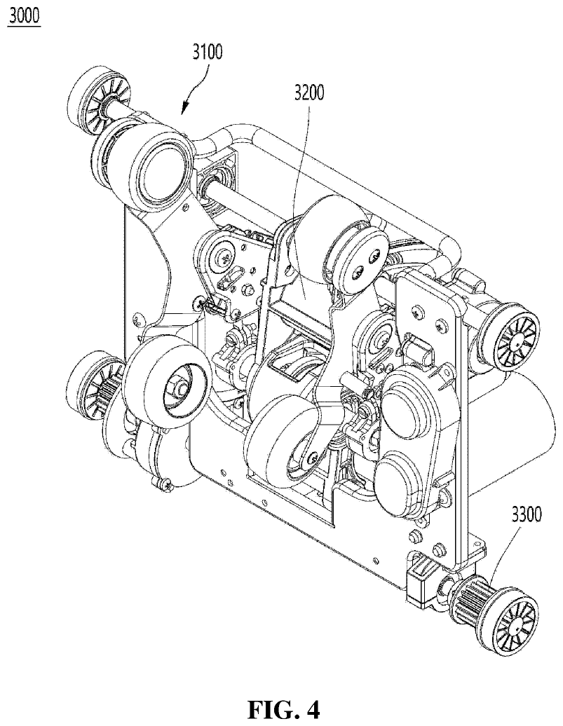

FIG. 4 is a view illustrating the body massage module according to one embodiment of the present disclosure.

Referring to FIG. 4, the body massage module 3000 according to one embodiment of the present disclosure may include at least one of a massage unit 3100, an actuator 3200, and a pinion gear 3300.

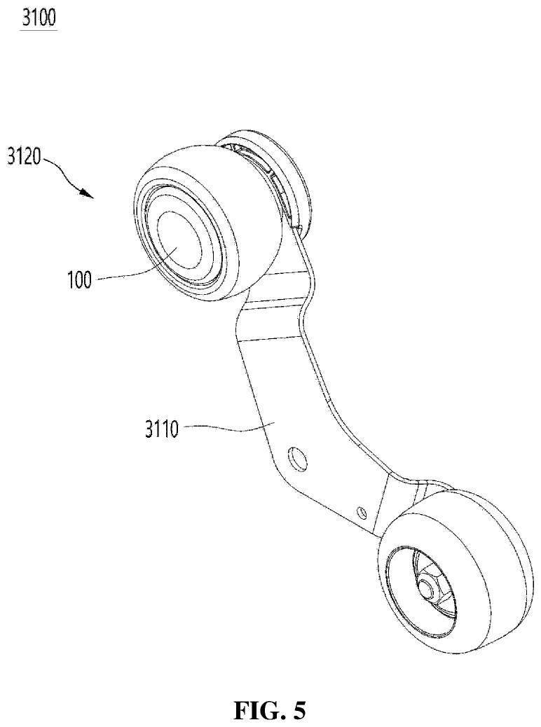

The massage unit 3100 may mechanically stimulate the user's body. Specifically, the massage unit 3100 may mechanically stimulate at least one of the user's head area, back area, and hip area. The massage unit 3100 according to one embodiment of the present disclosure may include a massage ball, and the massage ball may massage the user's body and provide a feeling of warmth at the same time. The detailed description will be described below with reference to FIGS. 5 to 12.

The pinion gear 3300 may move the body massage module 3000 along the rack gear of the main frame. The pinion gear 3300 may mesh with the rack gear and may move. The pinion gear 3300 may be rotated by the actuator 3200. As the pinion gear 3300 is rotated by the actuator 3200, the body massage module 3000 may move downward or upward along the rack gear. The actuator 3200 may move the body massage module 3000

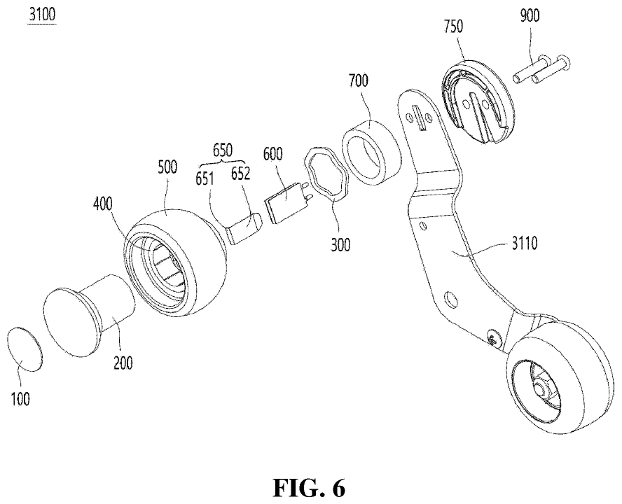

FIG. 5 is a perspective view illustrating an assembled massage unit according to a first embodiment of the present disclosure, and FIG. 6 is an exploded perspective view illustrating a disassembled message unit according to the first embodiment of the present disclosure.

Referring to FIGS. 5 and 6, a massage unit 3100 according to the first embodiment of the present disclosure may include a unit frame 3110 and one or more massage balls 3120 coupled to the unit frame 3110.

The unit frame 3110 may fix the massage ball 3120 so that the massage ball 3120 moves along a body massage module. The massage balls 3120 may be fixed to one end and the other end of the unit frame 3110 according to one embodiment, and a central portion of the unit frame 3110 may be fixed to the body massage module, but the present disclosure is not limited thereto. A shape of the unit frame 3110 may be changed according to the number of massage balls 3120 fixed to the unit frame 3110.

The massage ball 3120 may directly massage a user's body. The massage ball 3120 according to one embodiment of the present disclosure may stimulate the user's body and provide a feeling of warmth at the same time. The massage ball 3120 may be provided as a plurality of massage balls 3120. Only some of the massage balls 3120 according to one embodiment may also provide a warming function.

Referring to FIG. 6, the massage ball 3120 according to the first embodiment of the present disclosure may include at least one of an insulation cap 100, a support shaft member 200, an insert member 400, a cushion member 500, a heating member 600, a spring member 650, a washer member 300, a bushing member 700, a cover member 750, and a coupling member 900.

The insulation cap 100 may partially block heat generated by the heating member 600 from being emitted to the outside of the massage ball 3120. The insulation cap 100 may partially block internal components of the massage ball 3120 from the outside so that the heat is concentrated on the cushion member 500.

The insulation cap 100 according to one embodiment may be coupled to a lower surface of the support shaft member 200. A size of the insulation cap 100 according to one embodiment may be smaller than a size of the lower surface of the support shaft member 200. The insulation cap 100 according to one embodiment may be disposed to cover a central portion of the lower surface of the support shaft member 200. Accordingly, the insulation cap 100 may block heat from being emitted from the central portion of the lower surface of the support shaft member 200 to the outside, and an inner portion of the support shaft member 200 may be exposed to the outside. The insulation cap 100 according to one embodiment may be formed of a rubber material, plastic material, or the like of which thermal conductivity is low but is not limited thereto as long as it is a material capable of blocking heat radiation.

The support shaft member 200 may fix the heating member 600. The heating member 600 may be fixedly inserted into the support shaft member 200. The support shaft member 200 may transfer the heat transferred from the heating member 600 to the cushion member 500 through the insert member 400 circumscribing the support shaft member 200. The support shaft member 200 according to one embodiment may be formed of a metal material having high thermal conductivity.

In addition, the support shaft member 200 is exposed to the outside to directly transfer the heat transferred from the heating member 600 to the user. The user may receive a feeling of warmth from the lower surface of the support shaft member 200. The lower surface of the support shaft member 200 may laterally protrude further than a side surface thereof to prevent foreign matter from flowing into the massage ball 3120.

In addition, the support shaft member 200 may support the internal components in the massage ball 3120. The support shaft member 200 may be manufactured as an integrated structure having a flange including a first surface through which the internal components are inserted and a second surface positioned in a direction opposite to the first surface and having an area greater than an area of the first surface. The first surface may be the lower surface of the support shaft member 200, and the second surface may be the side surface thereof. The support shaft member 200 according to one embodiment may have a cylindrical shape, in which the lower surface protrudes further than the side surface, but is not limited thereto. At least one of the washer member 300, the insert member 400, the cushion member 500, and the bushing member 700 may be fitted to the side surface of the support shaft member 200. The support shaft member 200 may be coupled to the unit frame 3110 using the coupling member 900.

The insert member 400 may maintain a shape of the cushion member 500. The insert member 400 may be provided in the cushion member 500 to maintain the shape of the cushion member 500 which may be formed of a soft material. In addition, the insert member 400 may circumscribe and be fitted to the support shaft member 200 to couple the cushion member 500 to the support shaft member 200. The insert member 400 according to one embodiment may be formed of a metal material having high thermal conductivity.

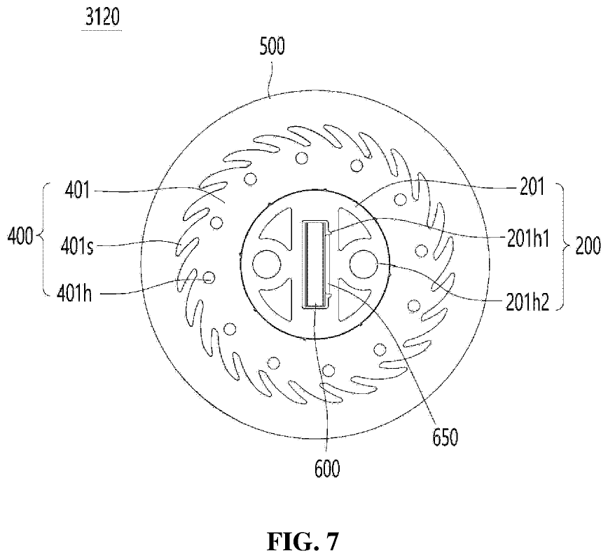

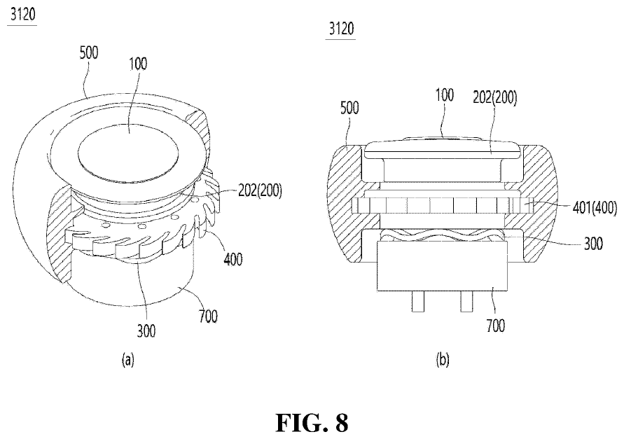

In addition, the insert member 400 may transfer the heat generated by the heating member 600 to the cushion member 500. The insert member 400 according to one embodiment of the present disclosure may include an insert gap passing through the insert member 400 and a protruding member in order to effectively transfer the heat to the cushion member 500. Detailed contents thereof will be described with reference to FIGS. 7 and 8.

The cushion member 500 may provide a feeling of a soft massage to the user. The cushion member 500 may directly massage the user at an outermost side of the massage ball 3120. The cushion member 500 according to one embodiment may be formed of a soft material and a material having high thermal conductivity but is not limited thereto.

In addition, the cushion member 500 may directly transfer the heat transferred from the heating member 600 to the user. The user may receive a feeling of warmth from the cushion member 500. The cushion member 500 according to one embodiment may be disposed to surround the insert member 400, and the shape of the cushion member 500 may be maintained by the insert member 400. The cushion member 500 according to one embodiment may be integrally formed with the insert member 400 by injection molding or the like, and the cushion member 500 may be disposed to surround the support shaft member 200 with the insert member 400 interposed therebetween and may receive the heat of the heating member 600 inserted into the support shaft member 200.

The heating member 600 may adjust a temperature of the massage ball 3120. Specifically, the heating member 600 may raise the temperature of the massage ball 3120 so that the massage ball 3120 becomes warm. The heating member 600 may be inserted into the support shaft member 200 disposed at an internal center thereof to uniformly transfer heat to the massage ball 3120. The heating member 600 as a heat source may transfer heat to the support shaft member 200, and the support shaft member 200 may transfer the heat to the cushion member 500 through the insert member 400. The heating member 600 according to one embodiment may be a positive temperature coefficient (PTC) heater but is not limited thereto. Meanwhile, a temperature of the heating member 600 according to one embodiment may be adjusted by a control unit but is not limited thereto.

The spring member 650 may fix the heating member 600 to the support shaft member 200. The heating member 600 may be inserted into a first member insertion groove provided in the support shaft member 200 and may not be pressed against the support shaft member 200 due to tolerance. Accordingly, in the massage ball 3120 according to the first embodiment of the present disclosure, the spring member 650 may be inserted between the heating member 600 and the support shaft member 200 to press the heating member 600 against an inner wall surface of the support shaft member 200.

The spring member 650 according to one embodiment of the present disclosure may include a first spring surface 651 in contact with a lower surface of the heating member 600 and a second spring surface 652 which extends from the first spring surface 651 and is in contact with a side surface of the heating member 600. An end of the second spring surface 652 may be bent in a direction toward the first spring surface 651 and have elasticity. When the spring member 650 is inserted into the first member insertion groove of the support shaft member 200 along with the heating member 600, the end of the second spring surface 652 may push and press the heating member 600 against the inner wall surface of the support shaft member 200.

The washer member 300 may adjust a gap between the support shaft member 200 and the insert member 400 and/or a gap between the insert member 400 and the bushing member 700. The washer member 300 according to one embodiment may have a circular ring shape and may be fitted to the side surface of the support shaft member 200 to be in contact with a lower surface of the insert member 400. One surface of the washer member 300 may be in contact with the insert member 400, and the other surface thereof may be in contact with the bushing member 700.

The washer member 300 may push the insert member 400 in a direction toward the lower surface of the support shaft member 200 to prevent the generation of a space between the support shaft member 200 and the insert member 400. The washer member 300 prevents the generation of the space between the support shaft member 200 and the insert member 400 so that the support shaft member 200 and the insert member 400 may be maintained to be close and heat of the support shaft member 200 may be efficiently transferred to the insert member 400. In addition, the washer member 300 may fill an empty space which may be generated between the insert member 400 and the bushing member 700.

In addition, the washer member 300 may serve to buffer the pressure applied to the massage ball 3120 by the user. The washer member 300 according to one embodiment may have a shape protruding upward and downward when viewed from the side. Specifically, the washer member 300 according to one embodiment may be repeatedly bent to have a wavy shape, a part of the washer member 300 is in contact with the insert member 400, and another part thereof may be in contact with the bushing member 700.

When the user's body is in contact with the massage ball 3120, while pressure is applied to the insert member 400, the insert member 400 may press the washer member 300. Since the washer member 300 according to one embodiment may have elasticity, a bent portion of the washer member 300 may be deformed due to the pressure, and all portions of the washer member 300 may be straightened so that the washer member 300 is in contact with the insert member 400 and the bushing member 700 at the same time. When the user's body is spaced apart from the massage ball 3120, the washer member 300 may be restored to an original state, and the washer member 300 may serve as a buffer while repeating deformation and restoration.

The bushing member 700 may insulate the heating member 600 from other components. In addition, the bushing member 700 may block heat generated by the heating member 600 from being emitted to the outside from an unnecessary portion. The bushing member 700 may block the internal components of the massage ball 3120 from the outside so that the heat is concentrated on the cushion member 500.

The bushing member 700 according to one embodiment may be formed in a hollow cylindrical shape but is not limited thereto. The bushing member 700 according to one embodiment may be disposed to surround the support shaft member 200 and may prevent heat of the support shaft member 200 from being emitted to the outside. In addition, the bushing member 700 may prevent foreign matter from flowing into the massage ball 3120. One side of the bushing member 700 according to one embodiment may be in contact with the washer member 300, the other side thereof may be in contact with the unit frame 3110, and the bushing member 700 may be fixed by the coupling member 900. The bushing member 700 according to one embodiment may be formed of a rubber material, plastic material, or the like having low thermal conductivity but is not limited thereto as long as it is a material capable of blocking heat radiation.

The cover member 750 may cover the heating member 600 to prevent separation of the heating member 600. In addition, the cover member 750 may prevent the heat generated by the heating member 600 from being emitted to the outside of the massage ball 3120. The cover member 750 according to one embodiment may be formed of a rubber material, plastic material, or the like having low thermal conductivity but is not limited thereto as long as it is a material capable of blocking heat radiation.

The coupling member 900 may fix the massage ball 3120 to the unit frame 3110. In addition, the coupling member 900 may couple the internal components of the massage ball 3120. The coupling member 900 according to one embodiment may couple at least one of the support shaft member 200, the bushing member 700, and the cover member 750. The coupling member 900 according to one embodiment may be formed as a screw but is not limited thereto.

FIG. 7 is a cross-sectional view illustrating the massage ball according to the first embodiment of the present disclosure when viewed from above, and FIG. 8 is a set of cross-sectional views illustrating the massage ball according to the first embodiment of the present disclosure when viewed from the side.

Referring to FIGS. 7 and 8, the massage ball 3120 according to the first embodiment of the present disclosure may include at least one of the insulation cap 100, the support shaft member 200, the washer member 300, the insert member 400, the cushion member 500, the heating member 600, the spring member 650, the bushing member 700, the cover member 750, and the coupling member 900. In FIGS. 7 and 8, the support shaft member 200 and the insert member 400 will be described in detail, and repeated descriptions thereof will be omitted.

The support shaft member 200 may have a columnar shape and include a side surface 201, to which internal components may be fitted, and a lower surface 202 serving as a cover.

A first member insertion groove 201h1, into which the heating member 600 may be inserted, and a second member insertion groove 201h2, into which the coupling member may be inserted, may be provided in the side surface 201.

The first member insertion groove 201h1 is a groove provided so that the heating member 600 may be inserted into the support shaft member 200, and a size of the first member insertion groove 201h1 may be greater than or equal to a size of the support shaft member 200. When the size of the first member insertion groove 201h1 is greater than the size of the support shaft member 200 due to manufacturing tolerance, the heating member 600 may be pressed against the inner wall surface of the support shaft member 200 by inserting the spring member 650 into the first member insertion groove 201h1 along with the heating member 600.

The spring member 650 is a member for fixing the heating member 600 to the first member insertion groove 201h1, and a central portion of the spring member 650 may be bent to push the heating member 600 so that the heating member 600 is in contact with the inner wall surface of the support shaft member 200.

The insert member 400 may have a hollow columnar shape and include a side surface protruding part 401. The side surface protruding part 401 is a part protruding from a side surface of the insert member 400. The side surface protruding part 401 may be inserted into the cushion member 500, may maintain the shape of the cushion member 500, and may transfer heat. The protruding member 401s and the insert gap 401h may be provided in the side surface protruding part 401 of the insert member 400.

The protruding member 401s may have a cogwheel shape, of which teeth are formed in a spiral shape, but is not limited thereto. The protruding member 401s is a heat radiation protrusion, may transfer heat transferred from the heating member 600 and the support shaft member 200 to the cushion member 500. In the protruding member 401s according to one embodiment, a protrusion is formed in a spiral shape, a contact area with the cushion member 500 may increase, and thus heat may be effectively transferred to the cushion member 500.

In addition, the protruding member 401s may have elasticity. When the user's body comes into contact with the massage ball 3120, the protruding member 401s may be deformed while pressure is applied to the cushion member 500 and the protruding member 401s, and the protruding member 401s may absorb impact. When the user's body is separated from the massage ball 3120, the protruding member 401s may be restored to an original state, and the protruding member 401s may serve as a buffer while repeating deformation and restoration.

The insert gap 401h may be a through hole passing through the side surface protruding part 401. The cushion member 500 may be inserted into the insert gap 401h, and thus, a coupling force between the cushion member 500 and the insert member 400 may be increased. Specifically, when the cushion member 500 is injected onto the insert member 400, the cushion member 500 may fill the insert gap 401h, and thus, a contact area between the cushion member 500 and the insert member 400 may be increased. Since the contact area between the cushion member 500 and the insert member 400 is increased, heat of the insert member 400 may be efficiently transferred to the cushion member 500. The insert gap 401h may be provided as one or more insert gaps 401h in the side surface protruding part 401 of the insert member 400.

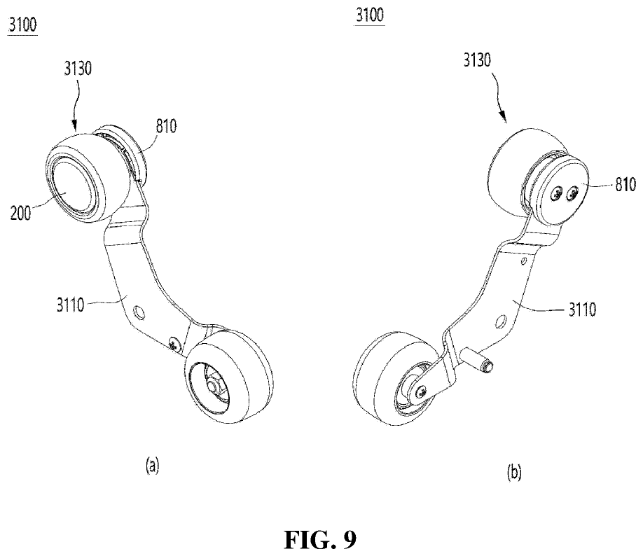

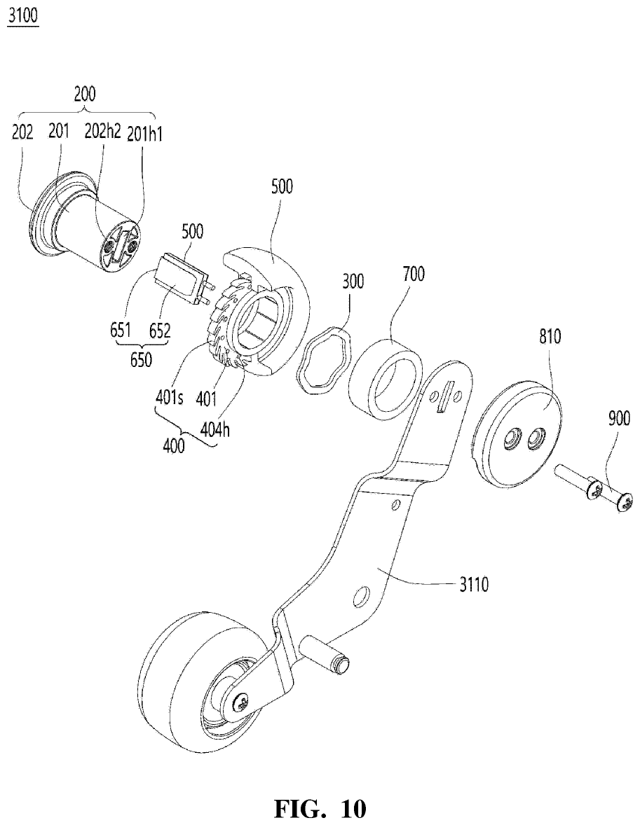

FIG. 9 is a set of perspective views illustrating an assembled massage unit according to a second embodiment of the present disclosure, and FIG. 10 is an exploded perspective view illustrating a disassembled massage unit according to the second embodiment of the present disclosure.

Referring to FIGS. 9 and 10, a massage unit 3100 according to the second embodiment of the present disclosure may include a unit frame 3110 and one or more massage balls 3130 coupled to the unit frame 3110.

The unit frame 3110 may fix the massage ball 3130 so that the massage ball 3130 may move along a body massage module. The massage balls 3130 may be fixed to one end and the other end of the unit frame 3110 according to one embodiment, and a central portion of the unit frame 3110 may be fixed to the body massage module but is not limited thereto. A shape of the unit frame 3110 may be changed according to the number of massage balls 3130 fixed to the unit frame 3110.

The massage ball 3130 may directly massage a user's body. The massage ball 3130 according to one embodiment of the present disclosure may stimulate the user's body and provide a feeling of warmth at the same time. The massage ball 3130 may be provided as a plurality of massage balls 3130. Only some of the massage balls 3130 according to one embodiment may also provide a warming function.

Referring to FIG. 10, the massage ball 3130 according to the second embodiment of the present disclosure may include at least one of a support shaft member 200, an insert member 400, a cushion member 500, a heating member 600, a spring member 650, a washer member 300, a bushing member 700, an outer warming member 810, and a coupling member 900. In the massage ball 3130 according to the second embodiment of the present disclosure, an insulation cap may not be provided, and the outer warming member 810 may be added thereto instead of the cover member unlike the massage ball according to the first embodiment.

The support shaft member 200 may fix the heating member 600. The heating member 600 may be fixedly inserted into the support shaft member 200. The support shaft member 200 may transfer the heat transferred from the heating member 600 to the cushion member 500 through the insert member 400. The support shaft member 200 according to one embodiment may be formed of a metal material having high thermal conductivity.

In addition, the support shaft member 200 may support internal components of the massage ball 3130. The support shaft member 200 according to one embodiment may have a cylindrical shape, in which a lower surface 202 protrudes further than a side surface 201, but is not limited thereto. At least one of the washer member 300, the insert member 400, the cushion member 500, and the bushing member 700 may be fitted to the side surface 201 of the support shaft member 200. The support shaft member 200 may be coupled to the unit frame 3110 using the coupling member 900.

The support shaft member 200 may have a columnar shape and include the side surface 201, to which internal components are fitted, and the lower surface 202 serving as a cover.

A first member insertion groove 201h1, into which the heating member 600 may be inserted, and a second member insertion groove 201h2, into which the coupling member may be inserted, may be provided in the side surface 201.

The first member insertion groove 201h1 is a groove provided so that the heating member 600 may be inserted into the support shaft member 200, and a size of the first member insertion groove 201h1 may be greater than or equal to a size of the support shaft member 200. When the size of the first member insertion groove 201h1 is greater than the size of the support shaft member 200 due to manufacturing tolerance, the heating member 600 may be pressed against an inner wall surface of the support shaft member 200 by inserting the spring member 650 into the first member insertion groove 201h1 along with the heating member 600.

The lower surface 202 may laterally protrude further than the side surface 201 to prevent foreign matter from flowing into the massage ball 3130. In addition, the lower surface 202 may be exposed to the outside to transfer heat transferred from the heating member 600 to a user. The user may receive a feeling of warmth from the lower surface 202 of the support shaft member 200. The insert member 400 may maintain a shape of the cushion member 500. The insert member 400 may be provided in the cushion member 500 to maintain the shape of the cushion member 500 which may be formed of a soft material. In addition, the insert member 400 may be fitted to the support shaft member 200 to couple the cushion member 500 to the support shaft member 200. The insert member 400 according to one embodiment may be formed of a metal material having high thermal conductivity.

In addition, the insert member 400 may transfer the heat generated by the heating member 600 to the cushion member 500. The insert member 400 according to one embodiment of the present disclosure may include an insert gap 401h, which passes through the insert member 400, and a protruding member 401s in order to effectively transfer heat to the cushion member 500. The insert member 400 may have a hollow columnar shape and include a side surface protruding part 401. The side surface protruding part 401 is a part protruding from a side surface of the insert member 400. The side surface protruding part 401 may be inserted into the cushion member 500, may maintain the shape of the cushion member 500, and may transfer heat. The protruding member 401s and the insert gap 401h may be provided in the side surface protruding part 401 of the insert member 400.

The protruding member 401s may have a cogwheel shape, of which teeth are formed in a spiral shape, but is not limited thereto. The protruding member 401s is a heat radiation protrusion, may transfer heat transferred from the heating member 600 and the support shaft member 200 to the cushion member 500. In the protruding member 401s, a protrusion is formed in a spiral shape, a contact area with the cushion member 500 may increase, and thus heat may be effectively transferred to the cushion member 500.

In addition, the protruding member 401s may have elasticity. When the user's body comes into contact with the massage ball 3130, the protruding member 401s may be deformed while pressure is applied to the cushion member 500 and the protruding member 401s, and the protruding member 401s may absorb impact. When the user's body is separated from the massage ball 3130, the protruding member 401s may be restored to an original state, and the protruding member 401s may serve as a buffer while repeating deformation and restoration.

The insert gap 401h may be a through hole passing through the side surface protruding part 401. The cushion member 500 may be inserted into the insert gap 401h, and a coupling force between the cushion member 500 and the insert member 400 may be increased. Specifically, when the cushion member 500 is injected onto the insert member 400, the cushion member 500 may fill the insert gap 401h, and thus, a contact area between the cushion member 500 and the insert member 400 may be increased. Since the contact area between the cushion member 500 and the insert member 400 is increased, heat of the insert member 400 may be efficiently transferred to the cushion member 500. The insert gap 401h may be provided as one or more insert gaps 401h in the side surface protruding part 401 of the insert member 400.

The cushion member 500 may provide a feeling of a soft massage to the user. The cushion member 500 may directly massage the user at an outermost side of the massage ball 3130. The cushion member 500 according to one embodiment may be formed of a soft material and a material having high thermal conductivity but is not limited thereto.

In addition, the cushion member 500 may directly transfer the heat transferred from the heating member 600 to the user. The user may receive a feeling of warmth from the cushion member 500. The cushion member 500 according to one embodiment may be disposed to surround the insert member 400, and the shape of the cushion member 500 may be maintained by the insert member 400. The cushion member 500 may be disposed to surround the support shaft member 200 with the insert member 400 interposed therebetween and may receive the heat of the heating member 600 inserted into the support shaft member 200.

The heating member 600 may adjust a temperature of the massage ball 3130. Specifically, the heating member 600 may raise the temperature of the massage ball 3130 so that the massage ball 3130 becomes warm. The heating member 600 may be inserted into the support shaft member 200 disposed at an internal center thereof to uniformly transfer heat to the massage ball 3130. The heating member 600 as a heat source may transfer heat to the support shaft member 200, and the support shaft member 200 may transfer the heat to the cushion member 500 through the insert member 400. The heating member 600 according to one embodiment may be a PTC heater but is not limited thereto. Meanwhile, a temperature of the heating member 600 according to one embodiment may be adjusted by a control unit but is not limited thereto.

The spring member 650 may fix the heating member 600 to the support shaft member 200. The heating member 600 may be inserted into a first member insertion groove 201h1 provided in the support shaft member 200 and may not be pressed against the support shaft member 200 due to tolerance. Accordingly, in the massage ball 3130 according to the second embodiment of the present disclosure, the spring member 650 may be inserted between the heating member 600 and the support shaft member 200 to press the heating member 600 against the inner wall surface of the support shaft member 200.

The spring member 650 according to one embodiment of the present disclosure may include a first spring surface 651 in contact with a lower surface of the heating member 600 and a second spring surface 652 which extends from the first spring surface 651 and is in contact with a side surface of the heating member 600. An end of the second spring surface 652 may be bent in a direction toward the first spring surface 651 and have elasticity. When the spring member 650 is inserted into the first member insertion groove 201h1 of the support shaft member 200 along with the heating member 600, the end of the second spring surface 652 may push and press the heating member 600 against the inner wall surface of the support shaft member 200.

The washer member 300 may adjust a gap between the support shaft member 200 and the insert member 400 and/or a gap between the insert member 400 and the bushing member 700. The washer member 300 according to one embodiment may have a circular ring shape and may be fitted to the side surface 201 of the support shaft member 200 to be in contact with a lower surface of the insert member 400. One surface of the washer member 300 may be in contact with the insert member 400, and the other surface thereof may be in contact with the bushing member 700.

The washer member 300 may push the insert member 400 in a direction toward the lower surface 202 of the support shaft member 200 to prevent the generation of a space between the support shaft member 200 and the insert member 400. The washer member 300 prevents the generation of the space between the support shaft member 200 and the insert member 400 so that the support shaft member 200 and the insert member 400 may be maintained to be close and heat of the support shaft member 200 may be efficiently transferred to the insert member 400. In addition, the washer member 300 may fill an empty space which may be generated between the insert member 400 and the bushing member 700.

In addition, the washer member 300 may serve to buffer the pressure applied to the massage ball 3130 by the user. The washer member 300 according to one embodiment may have a shape protruding upward and downward when viewed from the side. Specifically, the washer member 300 according to one embodiment may be repeatedly bent to have a wavy shape, a part of the washer member 300 is in contact with the insert member 400, and another part thereof may be in contact with the bushing member 700.

When the user's body is in contact with the massage ball 3130, while pressure is applied to the insert member 400, the insert member 400 may press the washer member 300. Since the washer member 300 according to one embodiment may have elasticity, a bent portion of the washer member 300 may be deformed due to the pressure, and all portions of the washer member 300 may be straightened so that the washer member 300 is in contact with the insert member 400 and the bushing member 700 at the same time. When the user's body is spaced apart from the massage ball 3130, the washer member 300 may be restored to an original state, and the washer member 300 may serve as a buffer while repeating deformation and restoration.

The bushing member 700 may insulate the heating member 600 from other components. In addition, the bushing member 700 may block heat generated by the heating member 600 from being emitted to the outside from an unnecessary portion.

The bushing member 700 according to one embodiment may be formed in a hollow cylindrical shape but is not limited thereto. The bushing member 700 according to one embodiment may be disposed to surround the support shaft member 200 and may block heat of the support shaft member 200 from being emitted to the outside. In addition, the bushing member 700 may prevent foreign matter from flowing into the massage ball 3130. One side of the bushing member 700 according to one embodiment may be in contact with the washer member 300, the other side thereof may be in contact with the unit frame 3110, and the bushing member 700 may be fixed by the coupling member 900. The bushing member 700 according to one embodiment may be formed of a rubber material, plastic material, or the like having low thermal conductivity but is not limited thereto as long as it is a material capable of blocking heat radiation. The outer warming member 810 may cover the heating member 600 to prevent separation of the heating member 600. In addition, the outer warming member 810 may directly transfer the heat transferred from the heating member 600 to the user. The user may receive a feeling of warmth from the outer warming member 810. The outer warming member 810 according to one embodiment may be disposed on a rear surface of the unit frame 3110. The outer warming member 810 according to one embodiment may be formed of a metal material having high thermal conductivity.

The coupling member 900 may fix the massage ball 3130 to the unit frame 3110. In addition, the coupling member 900 may couple the internal components of the massage ball 3130. The coupling member 900 according to one embodiment may couple at least one of the support shaft member 200, the bushing member 700, and the outer warming member 810. The coupling member 900 according to one embodiment may be formed as a screw but is not limited thereto.

The massage ball 3130 according to the second embodiment of the present disclosure may provide a feeling of warmth to the user using the support shaft member 200 and the outer warming member 810 in addition to the cushion member 500. Since a rate of temperature increase of the cushion member 500 is lower than a rate of temperature increase of each of the support shaft member 200 and the outer warming member 810, the massage ball 3130 according to the second embodiment of the present disclosure may provide a feeling of warmth to the user using the support shaft member 200 and the outer warming member 810.

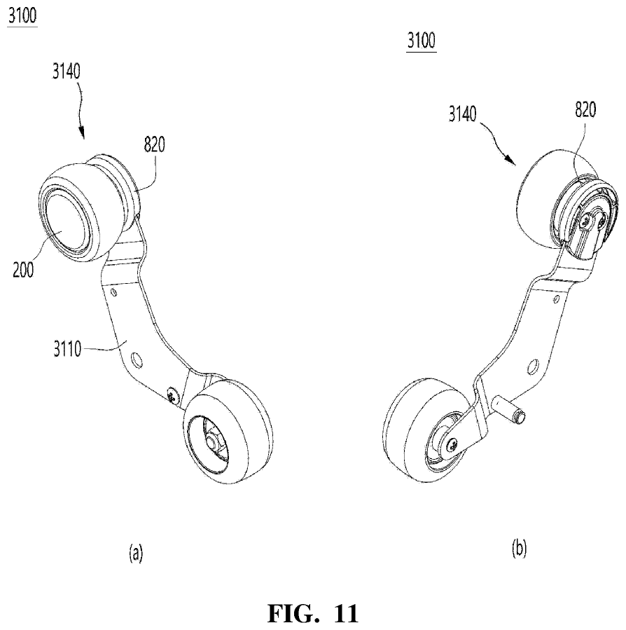

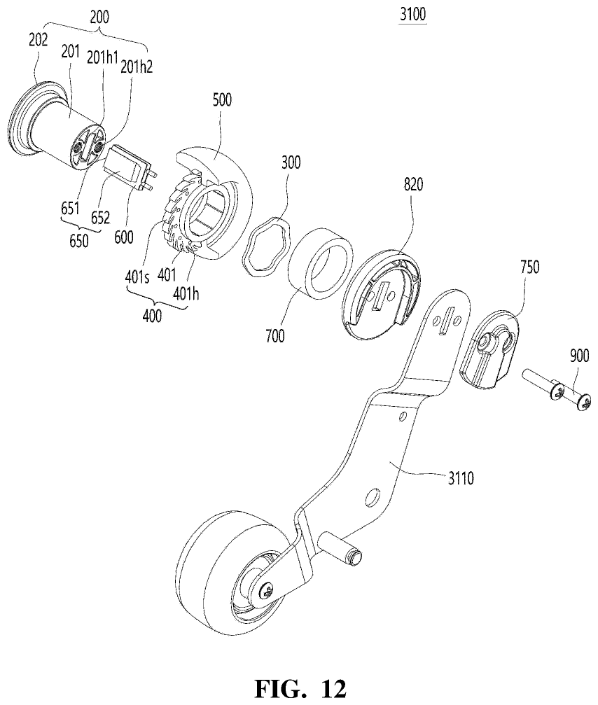

FIG. 11 is set of perspective views illustrating an assembled massage unit according to a third embodiment of the present disclosure, and FIG. 12 is an exploded perspective view illustrating a disassembled massage unit according to the third embodiment of the present disclosure.

Referring to FIGS. 11 and 12, a massage unit 3100 according to the third embodiment of the present disclosure may include a unit frame 3110 and one or more massage balls 3140 coupled to the unit frame 3110.

The unit frame 3110 may fix the massage ball 3140 so that the massage ball 3140 moves along a body massage module. The massage balls 3140 may be fixed to one end and the other end of the unit frame 3110 according to one embodiment, and a central portion of the unit frame 3110 may be fixed to the body massage module, but the present disclosure is not limited thereto. A shape of the unit frame 3110 may be changed according to the number of massage balls 3140 fixed to the unit frame 3110.

The massage ball 3140 may directly massage a user's body. The massage ball 3140 according to one embodiment of the present disclosure may stimulate the user's body and provide a feeling of warmth at the same time. The massage ball 3140 may be provided as a plurality of massage balls 3140. Only some of the massage balls 3140 according to one embodiment may also provide a warming function.

Referring to FIG. 12, the massage ball 3140 according to the third embodiment of the present disclosure may include at least one of a support shaft member 200, an insert member 400, a cushion member 500, a heating member 600, a spring member 650, a washer member 300, a bushing member 700, an inner warming member 820, a cover member 750, and a coupling member 900. In the massage ball 3140 according to the third embodiment of the present disclosure, the inner warming member 820 may be added instead of the outer warming member, and the cover member 750 may be added unlike the massage ball according to the second embodiment.

The support shaft member 200 may fix the heating member 600. The heating member 600 may be fixedly inserted into the support shaft member 200. The support shaft member 200 may transfer the heat transferred from the heating member 600 to the cushion member 500 through the insert member 400. The support shaft member 200 according to one embodiment may be formed of a metal material having high thermal conductivity.

In addition, the support shaft member 200 may support internal components of the massage ball 3140. The support shaft member 200 according to one embodiment may have a cylindrical shape in which a lower surface 202 protrudes further than a side surface 201 but is not limited thereto. At least one of the washer member 300, the insert member 400, the cushion member 500, the bushing member 700, and the inner warming member 820 may be fitted to the side surface 201 of the support shaft member 200. The support shaft member 200 may be coupled to the unit frame 3110 using the coupling member 900.

The support shaft member 200 may have a columnar shape and include the side surface 201, to which internal components are fitted, and the lower surface 202 serving as a cover.

A first member insertion groove 201h1, into which the heating member 600 may be inserted, and a second member insertion groove 201h2, into which the coupling member may be inserted, may be provided in the side surface 201.

The first member insertion groove 201h1 is a groove provided so that the heating member 600 may be inserted into the support shaft member 200, and a size of the first member insertion groove 201h1 may be greater than or equal to a size of the support shaft member 200. When the size of the first member insertion groove 201h1 is greater than the size of the support shaft member 200 due to manufacturing tolerance, the heating member 600 may be pressed against an inner wall surface of the support shaft member 200 by inserting the spring member 650 into the first member insertion groove 201h1 along with the heating member 600.

The lower surface 202 may laterally protrude further than the side surface 201 to prevent foreign matter from flowing into the massage ball 3140. In addition, the lower surface 202 may be exposed to directly transfer the heat transferred from the heating member 600 to a user. The user may receive a feeling of warm from the lower surface 202 of the support shaft member 200.

The insert member 400 may maintain a shape of the cushion member 500. The insert member 400 may be provided in the cushion member 500 to maintain the shape of the cushion member 500 which may be formed of a soft material. In addition, the insert member 400 may be fitted to the support shaft member 200 to couple the cushion member 500 to the support shaft member 200. The insert member 400 according to one embodiment may be formed of a metal material having high thermal conductivity.

In addition, the insert member 400 may transfer the heat generated by the heating member 600 to the cushion member 500. The insert member 400 according to one embodiment of the present disclosure may include an insert gap 401h, which passes through the insert member 400, and a protruding member 401s in order to effectively transfer heat to the cushion member 500.

The insert member 400 may have a hollow columnar shape and include a side surface protruding part 401. The side surface protruding part 401 is a part protruding from a side surface of the insert member 400. The side surface protruding part 401 may be inserted into the cushion member 500, may maintain the shape of the cushion member 500, and may transfer heat. The protruding member 401s and the insert gap 401h may be provided in the side surface protruding part 401 of the insert member 400.

The protruding member 401s may have a cogwheel shape, of which teeth are formed in a spiral shape, but is not limited thereto. The protruding member 401s is a heat radiation protrusion, may transfer heat transferred from the heating member 600 and the support shaft member 200 to the cushion member 500. In the protruding member 401s, a protrusion is formed in a spiral shape, a contact area with the cushion member 500 may increase, and thus heat may be effectively transferred to the cushion member 500.

In addition, the protruding member 401s may have elasticity. When the user's body comes into contact with the massage ball 3140, the protruding member 401s may be deformed while pressure is applied to the cushion member 500 and the protruding member 401s, and the protruding member 401s may absorb impact. When the user's body is separated from the massage ball 3140, the protruding member 401s may be restored to an original state, and the protruding member 401s may serve as a buffer while repeating deformation and restoration.

The insert gap 401h may be a through hole passing through the side surface protruding part 401. The cushion member 500 may be inserted into the insert gap 401h, and a coupling force between the cushion member 500 and the insert member 400 may be increased. Specifically, when the cushion member 500 is injected onto the insert member 400, the cushion member 500 may fill the insert gap 401h, and thus, a contact area between the cushion member 500 and the insert member 400 may be increased. Since the contact area between the cushion member 500 and the insert member 400 is increased, heat of the insert member 400 may be efficiently transferred to the cushion member 500. The insert gap 401h may be provided as one or more insert gaps 401h in the side surface protruding part 401 of the insert member 400.

The cushion member 500 may provide a feeling of a soft massage to the user. The cushion member 500 may directly massage the user at an outermost side of the massage ball 3140. The cushion member 500 according to one embodiment may be formed of a soft material and a material having high thermal conductivity but is not limited thereto.

In addition, the cushion member 500 may directly transfer the heat transferred from the heating member 600 to the user. The user may receive a feeling of warmth from the cushion member 500. The cushion member 500 according to one embodiment may be disposed to surround the insert member 400, and the shape of the cushion member 500 may be maintained by the insert member 400. The cushion member 500 may be disposed to surround the support shaft member 200 with the insert member 400 interposed therebetween and may receive the heat of the heating member 600 inserted into the support shaft member 200.

The heating member 600 may adjust a temperature of the massage ball 3140. Specifically, the heating member 600 may raise the temperature of the massage ball 3140 so that the massage ball 3140 becomes warm. The heating member 600 may be inserted into the support shaft member 200 disposed at an internal center thereof to uniformly transfer heat to the massage ball 3140. The heating member 600 as a heat source may transfer heat to the support shaft member 200, and the support shaft member 200 may transfer the heat to the cushion member 500 through the insert member 400. The heating member 600 according to one embodiment may be a PTC heater but is not limited thereto. Meanwhile, a temperature of the heating member 600 according to one embodiment may be adjusted by a control unit but is not limited thereto.

The spring member 650 may fix the heating member 600 to the support shaft member 200. The heating member 600 may be inserted into a first member insertion groove 201h1 provided in the support shaft member 200 and may not be pressed against the support shaft member 200 due to tolerance. Accordingly, in the massage ball 3140 according to the third embodiment of the present disclosure, the spring member 650 may be inserted between the heating member 600 and the support shaft member 200 to press the heating member 600 against the inner wall surface of the support shaft member 200.

The spring member 650 according to one embodiment of the present disclosure may include a first spring surface 651 in contact with a lower surface of the heating member 600 and a second spring surface 652 which extends from the first spring surface 651 and is in contact with a side surface of the heating member 600. An end of the second spring surface 652 may be bent in a direction toward the first spring surface 651 and have elasticity. When the spring member 650 is inserted into the first member insertion groove 201h1 of the support shaft member 200 along with the heating member 600, the end of the second spring surface 652 may push and press the heating member 600 against the inner wall surface of the support shaft member 200.

The washer member 300 may adjust a gap between the support shaft member 200 and the insert member 400 and/or a gap between the insert member 400 and the bushing member 700. The washer member 300 according to one embodiment may have a circular ring shape and may be fitted to the side surface 201 of the support shaft member 200 to be in contact with a lower surface of the insert member 400. One surface of the washer member 300 may be in contact with the insert member 400, and the other surface thereof may be in contact with the bushing member 700.

The washer member 300 may push the insert member 400 in a direction toward the lower surface 202 of the support shaft member 200 to prevent the generation of a space between the support shaft member 200 and the insert member 400. The washer member 300 prevents the generation of the space between the support shaft member 200 and the insert member 400 so that the support shaft member 200 and the insert member 400 may be maintained to be close and heat of the support shaft member 200 may be efficiently transferred to the insert member 400. In addition, the washer member 300 may fill an empty space which may be generated between the insert member 400 and the bushing member 700.

In addition, the washer member 300 may serve to buffer the pressure applied to the massage ball 3140 by the user. The washer member 300 according to one embodiment may have a shape protruding upward and downward when viewed from the side. Specifically, the washer member 300 according to one embodiment may be repeatedly bent to have a wavy shape, a part of the washer member 300 is in contact with the insert member 400, and another part thereof may be in contact with the bushing member 700.

When the user's body is in contact with the massage ball 3140, while pressure is applied to the insert member 400, the insert member 400 may press the washer member 300. Since the washer member 300 according to one embodiment may have elasticity, a bent portion of the washer member 300 may be deformed due to the pressure, and all portions of the washer member 300 may be straightened so that the washer member 300 is in contact with the insert member 400 and the bushing member 700 at the same time. When the user's body is spaced apart from the massage ball 3140, the washer member 300 may be restored to an original state, and the washer member 300 may serve as a buffer while repeating deformation and restoration.

The bushing member 700 may insulate the heating member 600 from other components. In addition, the bushing member 700 may block heat generated by the heating member 600 from being emitted to the outside from an unnecessary portion.

The bushing member 700 according to one embodiment may be formed in a hollow cylindrical shape but is not limited thereto. The bushing member 700 according to one embodiment may be disposed to surround the support shaft member 200 and may prevent heat of the support shaft member 200 from being emitted to the outside. In addition, the bushing member 700 may prevent foreign matter from flowing into the massage ball 3140. One side of the bushing member 700 according to one embodiment may be in contact with the washer member 300, the other side thereof may be in contact with the inner warming member 820, and the bushing member 700 may be fixed by the coupling member 900. The bushing member 700 according to one embodiment may be formed of a rubber material, plastic material, or the like having low thermal conductivity but is not limited thereto as long as it is a material capable of blocking heat radiation.

The inner warming member 820 may directly transfer the heat transferred from the heating member 600 to the user. The user may receive a feeling of warmth from the inner warming member 820. The inner warming member 820 according to one embodiment may be disposed on a front surface of the unit frame 3110. The inner warming member 820 according to one embodiment may be formed of a metal material having high thermal conductivity.

The cover member 750 may cover the heating member 600 to prevent separation of the heating member 600. In addition, the cover member 750 may prevent the heat generated by the heating member 600 from being emitted to the outside of the massage ball 3140. The cover member 750 according to one embodiment may be formed of a rubber material, plastic material, or the like having low thermal conductivity but is not limited thereto as long as it is a material capable of blocking heat radiation.

The coupling member 900 may fix the massage ball 3140 to the unit frame 3110. In addition, the coupling member 900 may couple the internal components of the massage ball 3140. The coupling member 900 according to one embodiment may couple at least one of the support shaft member 200, the bushing member 700, the inner warming member 820, and the cover member 750. The coupling member 900 according to one embodiment may be formed as a screw but is not limited thereto.

The massage ball 3140 according to the third embodiment of the present disclosure may provide a feeling of warmth to the user using the support shaft member 200 and the inner warming member 820 in addition to the cushion member 500. Since a rate of temperature increase of the cushion member 500 is lower than a rate of temperature increase of each of the support shaft member 200 and the inner warming member 820, the massage ball 3140 according to the third embodiment of the present disclosure may provide a feeling of warmth to the user using the support shaft member 200 and the inner warming member 820.

Since the massage ball 3140 according to the third embodiment of the present disclosure includes the inner warming member 820, the massage ball 3140 can transfer heat more efficiently than the massage ball according to the second embodiment including the outer warming member.

Specifically, since the inner warming member 820 is disposed closer to the heating member 600 than the outer warming member disposed on a rear surface of the unit frame 3110, the inner warming member 820 can transfer heat more efficiently.

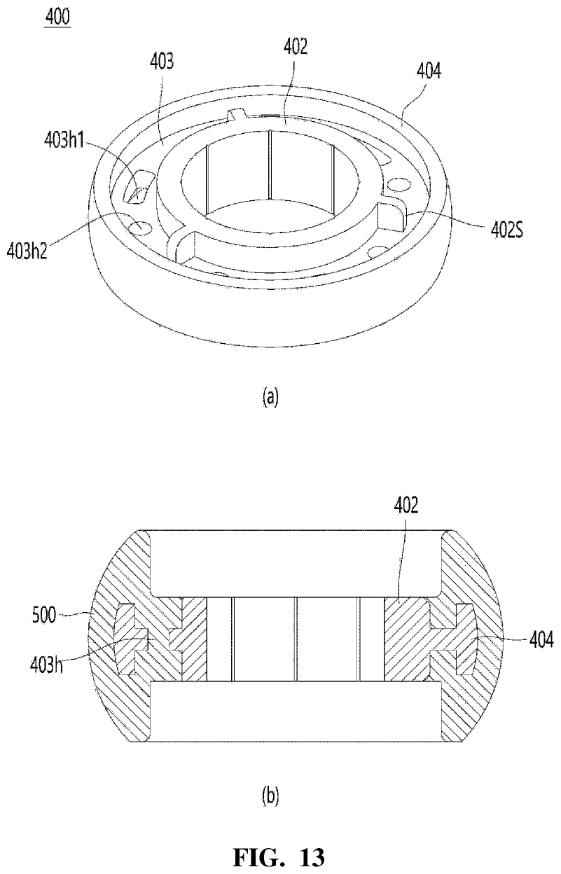

FIG. 13 is a set of views illustrating an insert member according to another embodiment of the present disclosure.

Specifically, FIG. 13A is a perspective view illustrating an insert member 400 according to another embodiment, and FIG. 13B is a cross-sectional view illustrating a shape in which the insert member 400 and a cushion member 500 according to another embodiment are coupled.

Referring to FIG. 13, the insert member 400 according to another embodiment of the present disclosure may include at least one of an inner edge protruding part 402, an insert groove part 403, and an outer protruding part 404.

The inner edge protruding part 402 is a protruding part capable of surrounding a support shaft member inserted into the insert member 400. The inner edge protruding part 402 may surround a hole passing through the insert member 400. The inner edge protruding part 402 may include one or more inner edge protrusions 402s. The inner edge protrusion 402s according to one embodiment may protrude from the inner edge protruding part 402 toward the insert groove part 403 but is not limited thereto. The inner edge protrusion 402s may increase a contact area between the insert member 400 and the cushion member 500 to increase a coupling force and transfer heat quickly.

The insert groove part 403 is a groove provided so that the cushion member 500 may be inserted into the insert member 400. The insert groove part 403 may be concavely provided between the inner edge protruding part 402 and the outer protruding part 404. The cushion member 500 may be provided to fill the insert groove part 403.

The insert groove part 403 may include one or more insert holes 403h1 and/or one or more insert gaps 403h2. The insert hole 403h1 is a hole provided so that the cushion member 500 may be inserted into the insert member 400. The insert hole 403h1 may pass through the insert groove part 403. The cushion member 500 may be provided to fill an inner portion of the insert hole 403h1. The insert hole 403h1 may increase a contact area between the insert member 400 and the cushion member 500 to increase a coupling force and transfer heat quickly.

The insert gap 403h2 may be a fine through hole passing through the insert groove part 403. The cushion member 500 may be inserted into the insert gap 403h2, and thus, a coupling force between the cushion member 500 and the insert member 400 may be increased. Specifically, when the cushion member 500 is injected onto the insert member 400, the cushion member 500 may fill an inner portion of the insert gap 403h2, and thus, a contact area between the cushion member 500 and the insert member 400 may be increased. Since the contact area between the cushion member 500 and the insert member 400 is increased, heat of the insert member 400 may be efficiently transferred to the cushion member 500. The insert gap 403h2 may be provided as one or more insert gaps 403h2 in the insert groove part 403.

The outer protruding part 404 may serve as a step configured to prevent the cushion member 500 inserted into the insert groove part 403 from falling out of the insert groove part 403. The outer protruding part 404 may be surrounded by the cushion member 500 but is not limited thereto.

The insert member 400 according to another embodiment of the present disclosure may be applied to each of the massage ball 3120 according to the first embodiment, the massage ball 3130 according to the second embodiment, and massage ball 3140 according to the third embodiment, which are described above.

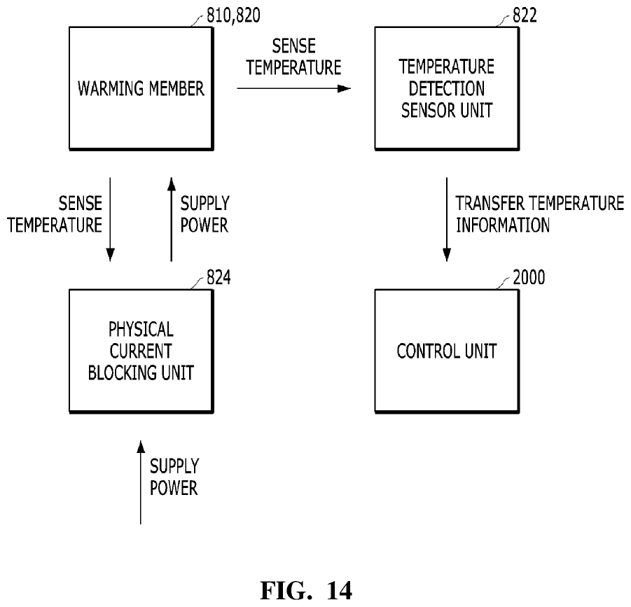

FIG. 14 is a view for describing a method of controlling a temperature of a massage ball according to another embodiment of the present disclosure.

According to one embodiment of the present disclosure, a temperature detection sensor unit 822 may be provided at the outer warming member 810 or the inner warming member 820. The temperature detection sensor unit 822 may sense a temperature of the warming member 810 or 820 and transmit sensed temperature information to the control unit 2000.

The control unit 2000 may control a current supplied to the heating member 600 on the basis of the received temperature information. For example, when the temperature information obtained from the temperature detection sensor unit 822 is greater than a predetermined threshold value, the control unit 2000 may block the current being supplied to the heating member 600.

According to one embodiment of the present disclosure, the outer warming member 810 or the inner warming member 820 may include a physical current blocking unit 824 which physically blocks the current being supplied to the heating member 600 according to the temperature of the outer warming member 810 or the inner warming member 820. In this case, the physical current blocking unit 824 may be implemented as a bi-metal module. The bi-metal module is a module which uses a difference in reaction between two metals according to heat and may serve as a kind of circuit breaker.

For example, the current supplied to the heating member 600 may flow through the physical current blocking unit 824 and may be supplied to the heating member 600, and when the temperature of the outer warming member 810 or the inner warming member 820 is higher than the predetermined threshold value, the physical current blocking unit 824 may physically block the current being supplied to the heating member 600 to prevent an accident in which a temperature of the massage ball increases abnormally.

According to one embodiment of the present disclosure, since the massage apparatus 1000 includes both of the temperature detection sensor unit 822 and the physical current blocking unit 824, the temperature of the massage ball is doubly prevented from increasing abnormally. For example, the control unit 2000 primarily controls the heating member 600 so that the temperature of the massage ball does not increase abnormally using the temperature information obtained from the temperature detection sensor unit 822. In this case, although there is a risk in which the control unit 2000 operates abnormally to increase the temperature of the heating member 600 abnormally, and even when the control unit 2000 operates abnormally, the physical current blocking unit 824 may physically block the current being supplied to the heating member 600 so that the temperature of the massage ball does not increase abnormally.

To this end, a threshold value applied to the control unit 2000 may be lower than a threshold value applied to the physical current blocking unit 824.

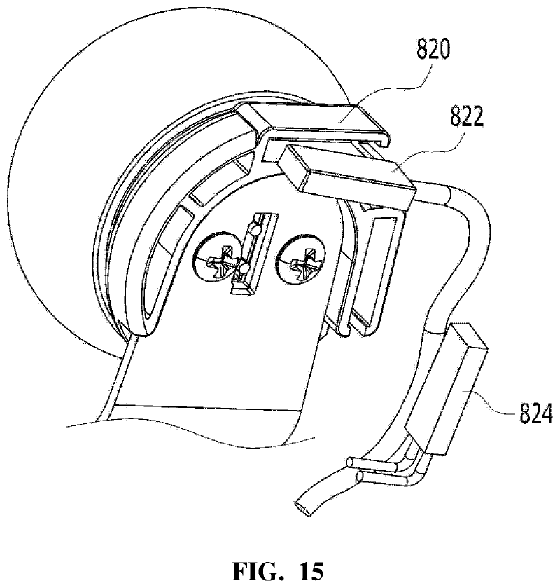

FIG. 15 is a view for describing the inner warming member 820 in which the temperature detection sensor unit 822 and the physical current blocking unit 824 are provided according to one embodiment of the present disclosure.

According to one embodiment of the present disclosure, the inner warming member 820 may include a plurality of grooves, and each of the temperature detection sensor unit 822 and the physical current blocking unit 824 may be provided in one hole.

Although the embodiments of the present disclosure have been described in detail with reference to the accompanying drawings, the present disclosure is not necessarily limited to the embodiments and various modifications may be made without departing from the technical spirit of the present disclosure. Therefore, the embodiments of the present disclosure are considered in a descriptive sense only and not for purposes of limitation. Therefore, the above-described embodiments should be considered as only illustrative in all aspects and not for purposes of limitation. The scope of the present disclosure should be interpreted by the appended claims and encompass all technical ideas falling within the scope of the appended claims.

1. A massage apparatus comprising a control unit and a body massage module which provides a massage function to at least a part of a user's body,
wherein the body massage module includes an actuator, which moves the body massage module, and a massage unit which mechanically stimulates the user's body,
the massage unit includes a massage ball which provides a feeling of warmth to a user,
the massage ball includes a heating member which adjusts a temperature of the massage ball, a support shaft member which fixes the heating member, a cushion member which surrounds the support shaft member and provides a feeling of a soft massage to the user, an insert member which maintains a shape of the cushion member, an inner warming member which receives heat from the heating member, and a temperature detection sensor unit which senses a temperature of the inner warming member, and
the control unit controls a temperature of the heating member on the basis of temperature information obtained from the temperature detection sensor unit.

2. The massage apparatus of claim 1, wherein, when the temperature information obtained from the temperature detection sensor unit is greater than a predetermined threshold value, the control unit blocks power being supplied to the heating member.

3. The massage apparatus of claim 2, further comprising a physical current blocking unit which is provided at the inner warming member and blocks a current being supplied to the heating member according to the temperature of the inner warming member.

4. The massage apparatus of claim 3, wherein the physical current blocking unit is formed as a bi-metal module.

5. The massage apparatus of claim 4, wherein the predetermined threshold value applied to the physical current blocking unit is higher than a predetermined threshold value used in the control unit.

6. A massage apparatus comprising a massage unit which stimulates a user's body and provides a feeling of warmth,
wherein the massage unit includes a unit frame and a massage ball coupled to the unit frame, and
the massage ball includes a support shaft member, which is coupled to the unit frame using a coupling member, a heating member fixedly inserted into the support shaft member, an insert member, which circumscribes the support shaft member and to which heat is transferred from the support shaft member, and a cushion member which surrounds the insert member and is integrally formed with the insert member.

7. The massage apparatus of claim 6, wherein the insert member includes:
an inner edge protruding part which surrounds the support shaft member;
an insert groove part provided so that the cushion member is inserted into the insert member; and
an outer protruding part which prevents separation of the cushion member inserted into the insert groove part.

8. The massage apparatus of claim 7, wherein:
the insert groove part further includes at least one through hole filled with a part of the cushion member; and
the through hole increases a contact area between the insert member and the cushion member to increase a coupling force and a heat transfer rate.

9. The massage apparatus of claim 7, wherein:
the inner edge protruding part further includes at least one inner edge protrusion protruding toward the insert groove part; and
the inner edge protrusion increases a contact area between the insert member and the cushion member to increase a coupling force and a heat transfer rate.

10. The massage apparatus of claim 6, wherein:
the support shaft member includes a first surface, into which the heating member is inserted and which is coupled to the unit frame, and a second surface which is positioned in a direction opposite to the first surface and has an area greater than an area of the first surface; and
the first surface further includes a first member insertion groove, into which the heating member is inserted, and a second member insertion groove into which the coupling member is inserted.
